(19) United States
(12) Patent Application Publication
(10) Pub. No.: US 2012/0282211 A1
(43) Pub. Date: Nov. 8, 2012

(54) ANTIBODIES AND CONJUGATES FOR MODULATORS OF ANGIOGENESIS

(75) Inventors: Newell R. Washburn, Pittsburgh, PA (US); Liang Tso Sun, Pittsburgh, PA (US)

(73) Assignee: CARNEGIE MELLON UNIVERSITY, Pittsburgh, PA (US)

(21) Appl. No.: 13/511,598

(22) PCT Filed: Nov. 24, 2010
(86) PCT No.: PCT/US10/58044

§ 371 Date: May 23, 2012

Related U.S. Application Data
(60) Provisional application No. 61/281,896, filed on Nov. 24, 2009.

(51) Int. Cl. C07K 17/10; C07K 17/08; C07K 19/00; C07K 17/12; C08B 37/06; A61K 38/39; C08B 37/08; A61K 39/395; A61K 31/75; A61K 38/02; A61K 31/7088; A61P 27/02; C07H 21/04; C08B 37/10

(52) U.S. Cl. 424/78.29; 530/391.1; 536/23.1; 530/300; 530/356; 536/2; 530/354; 536/21; 536/20; 530/350; 424/178.1; 424/78.27; 514/20.8; 514/44 R; 514/17.2; 525/54.1

(57) ABSTRACT

We provide methods and compositions for the treatment of dysregulation of blood vessel growth by regulation of neovascularization. Embodiments accomplish this by restricting the diffusion and transport of therapeutic agents through conjugating them to polymers or polymer constructs while retaining the binding affinities and functions of the therapeutic agents.

### CROSS-REFERENCE TO RELATED APPLICATIONS

This application claims priority to U.S. Provisional Patent Application No. 61/281,896, filed on Nov. 24, 2009. That application is incorporated by reference as if fully rewritten herein.

### STATEMENT REGARDING FEDERALLY SPONSORED RESEARCH OR DEVELOPMENT

This invention was made with government support under Army Grant No. W81XWH-08-2-0032. The government has certain rights in this invention.

### BACKGROUND OF THE INVENTION

1. Field of the Invention

Embodiments of the invention relate to modulators of angiogenesis and blood vessel formation and maintenance.

2. Background of the Related Art

Neovascularization involves the growth of immature blood vessels from surrounding vasculature. While important in normal tissue maintenance and development, neovascularization is a critical component of many disease states, such as age-related and wet macular degeneration, growth of malignant tumors, rheumatoid arthritis, and psoriasis. This process is driven by a host of soluble signaling molecules, such as vascular endothelial growth factors (VEGF), platelet-derived growth factors (PDGF), placental growth factor (PGF), and fibroblast growth factors (FGF). Other factors have been found to work in concert with VEGF to regulate vascular formation.

Blood vessel formation in adult tissues follows a cascade of specific events that are regulated by several soluble mediators. Carmeliet P., Mechanisms of angiogenesis and arteriogenesis. Nat Med. 2000; 6(4):389-95; Yancopoulos G D, Davis S, Gale N R, Rudge J S, Wiegand S J, Holash J. Vascular-specific growth factors and blood vessel formation. Nature. 2000; 407(6801):242-8. First, mature vasculature is stabilized by angiopoietin-1 (Ang1), which promotes interactions between endothelial cells and surrounding supporting cells, such as smooth muscle cells and pericytes. Then angiopoietin-2 (Ang2) destabilizes blood vessels, which can undergo angiogenic sprouting upon activation by VEGF or regression without VEGF signal.

Of the families of proteins involved in blood vessel formation, VEGF in particular has been used effectively as a therapeutic target. Recent research suggests, however, that there are numerous therapeutic targets, such as human protein tyrosine phosphatase beta (HPTP□). Examples of VEGF inhibitors include humanized antibodies AVASTIN® (bevacizumab; Genentech/Roche) and LUCENTIS® (ranibizumab; Genentech), and the RNA aptamer MACUGEN® (pegaptanib; OSI Pharmaceuticals/Pfizer). Other relevant therapeutic strategies include the fusion protein VEGF Trap-Eye® (aflibercept; Regeneron) and the inhibitor of HPTP□ currently being tested by Akebia Therapeutics for decreasing Ang2 activities. These inhibitors are applied locally or systemically and but can be cleared on the time scale of days or weeks, reducing their efficacy and increasing the cost.

Covalent conjugation of poly(ethylene glycol) (PEG) to therapeutic molecules is generally performed to increase the circulation half-life. A relevant example of PEGylated biomolecules is pegaptanib (brand name MACUGEN®), a PEGylated aptamer that binds VEGF165. Ng E. W., et al., “Pegaptanib, a targeted anti-VEGF aptamer for ocular vascular disease” Nat Rev Drug Discov. 2006; 5(2):123-32. Pegaptanib is composed of 27 nucleotides, and conjugation to a 40 kDa dimeric PEG reduces the rate of clearance of the drug. PEGylation decreased the binding affinity four-fold, but this is offset by the reductions in clearance rates. Veronese & Mero, “The impact of PEGylation on biological therapies” BioDrugs. 2008; 22(5)315-29.

Conjugation of inhibitors of pro-inflammatory cytokines to high molecular weight polysaccharides has been shown to be an effective strategy for localizing their activities. Constructs composed of monoclonal antibodies against interleukin-1β or tumor necrosis factor-α conjugated to hyaluronic acid or carboxymethylcellulose retain their binding affinities, Sun, et al., “Cytokine Binding by Polysaccharide-Antibody Conjugates” Mol Pharm. 2010, and are active in vivo. Sun, et al. “Biological activities of cytokine-neutralizing hyaluronic acid-antibody conjugates” Wound Repair Regen, 2010; 18(3)302-10. When cross-linked into a solid gel, the antibodies are still capable of binding cytokines but the solid conjugates are no longer effective at controlling inflammation, which may be due to the long diffusion path into the solid gel. Sun, et al., “Design principles for cytokine-neutralizing gels: Cross-linking effects” Acta Biomater. 2010.

Conjugates composed of inhibitors of pro-inflammatory cytokines that are conjugated to polymers and polymer constructs were the subject of PCT International Application No. PCT/US2008/073335, filed on Aug. 15, 2008, and incorporated by reference herein. That work reports that inhibitors of interleukin-1β and tumor necrosis-factor-α were still biologically active even after conjugation to a diversity of polysaccharides. However, given the dissimilar compositions and structures of mediators of angiogenesis, there is no guarantee that polymer constructs of their inhibitors would retain their binding affinities. In addition, inflammation is generally a condition which, if fully resolved, will not spontaneously revert back to its original state. In contrast, most conditions characterized by dysregulation of blood vessel formation or maintenance present an underlying disease state, as in the case of cancerous tumors, that has fundamental angiogenic tendencies, making treatment of these conditions with polymer-conjugated inhibitors distinctly different from treating inflammatory conditions with an analogous strategy.

### BRIEF SUMMARY OF THE INVENTION

Dysregulation of blood vessel growth and development is a critical component of disease states ranging from tumorigenesis in cancer to wet macular degeneration. The process of neovascularization is regulated by soluble signaling molecules, such as vascular endothelial growth factors, platelet-derived growth factors, and fibroblast growth factors. Recombinant proteins, aptamers, or other molecules that neutralize these angiogenic factors are used to treat these conditions, but their efficacy is limited by poor targeting to the areas where they are needed. The technology described in this application is engineered to provide sustained action of compounds that regulate neovascularization and blood vessel maintenance at the site of delivery.

We provide methods and compositions for the treatment of dysregulation of blood vessel growth by regulation of neovascularization. Embodiments accomplish this by restricting the diffusion and transport of therapeutic agents through conjugating them to polymers or polymer constructs while retaining the binding affinities and functions of the therapeutic agents.

### BRIEF DESCRIPTION OF THE FIGURES

FIG. 1 shows a schematic representation of (anti-VEGF)-hyaluronic acid construct.

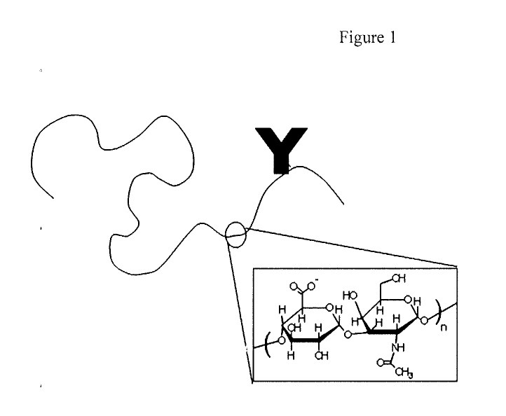

FIG. 2 shows a polyacrylamide gel electrophoresis (PAGE) assay using Alcian Blue staining, (i, ii) 0.1×HA-antiVEGF construct, (iii) 0.03% wt HA, (iv) 0.06% wt HA, (v) 0.12% wt HA, (vi) 0.25% wt HA, and (vii) 0.5% wt HA.

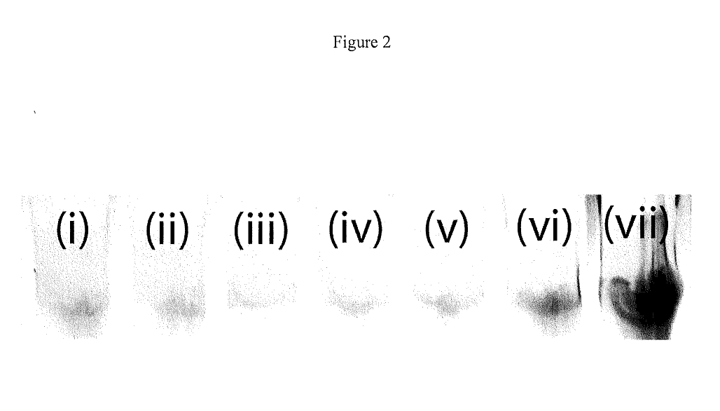

FIG. 3 shows a standard curve of HA quantification results based on PAGE data.

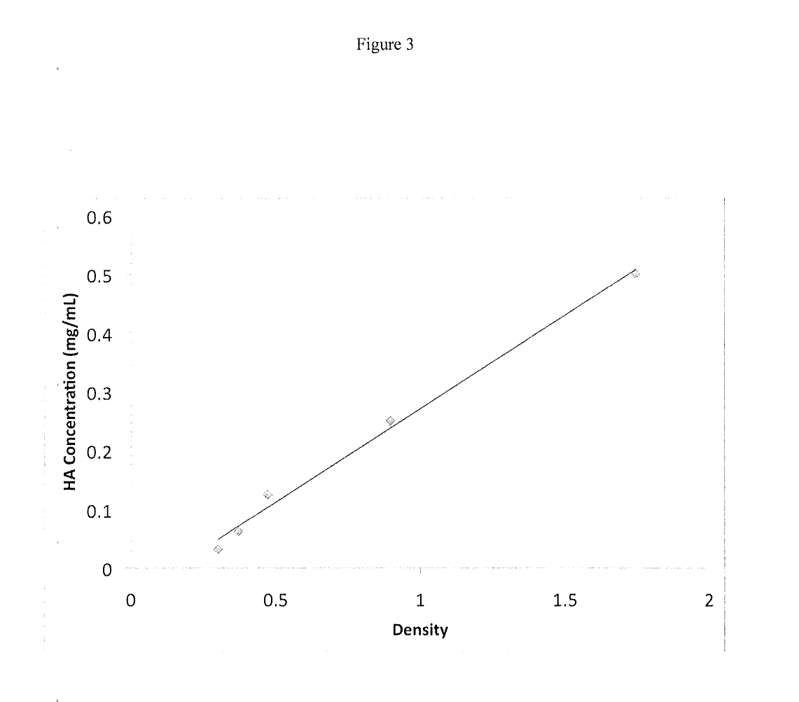

FIG. 4 shows a standard curve of antibody quantification results using fluorescence immunosorbent assay.

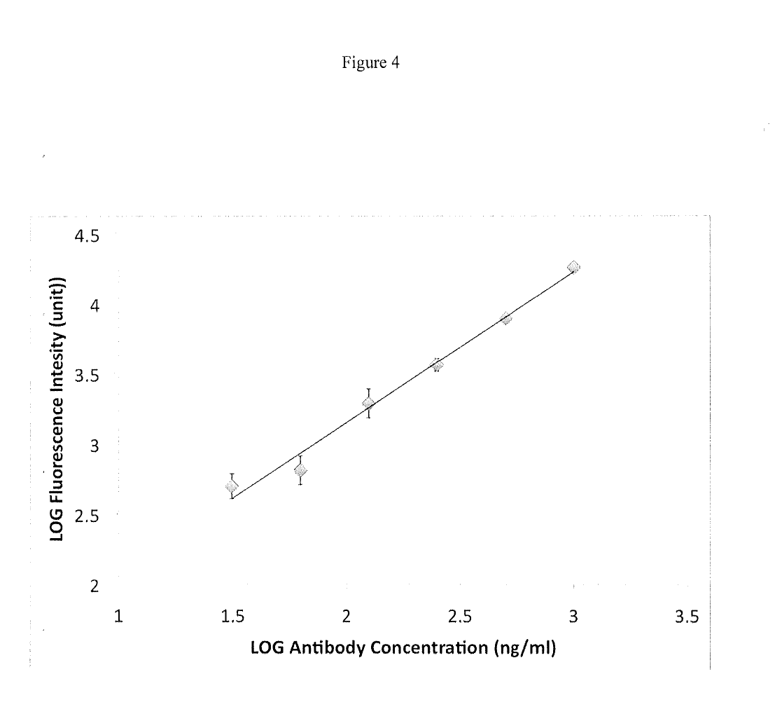

FIG. 5 shows association and dissociation curves of anti-VEGF and HA-anti-VEGF binding to rhVEGF using ForteBio Octet system. The curves lighter in color are the best-fit curves used for quantitative analysis, which overlap with data points.

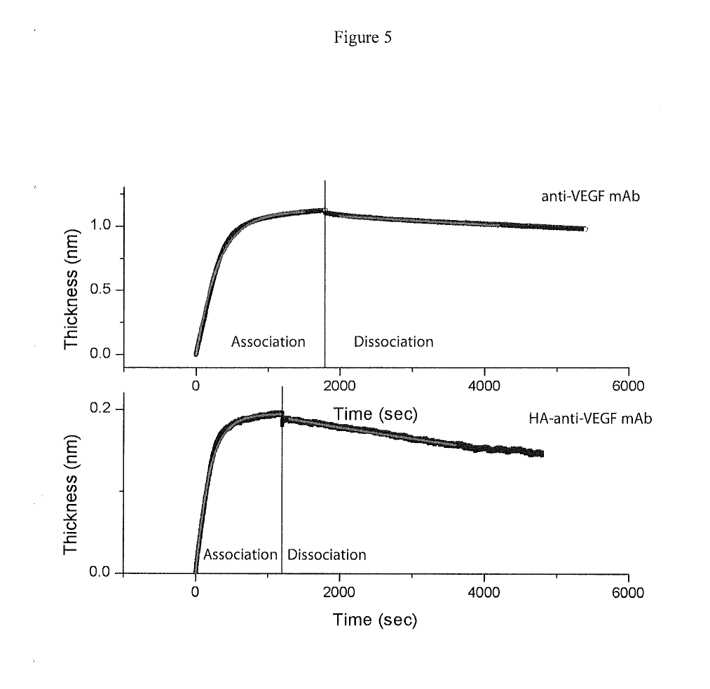

FIG. 6 shows masson trichrome staining of CAM tissues stimulated by collagen constructs with different agents incorporated, (a) anti-VEGF mAb, (b) HA, (c) HA-anti-VEGF conjugate, (d) rhVEGF. Asterisk indicates the location of the collagen constructs, and arrows indicate the observed vasculatures.

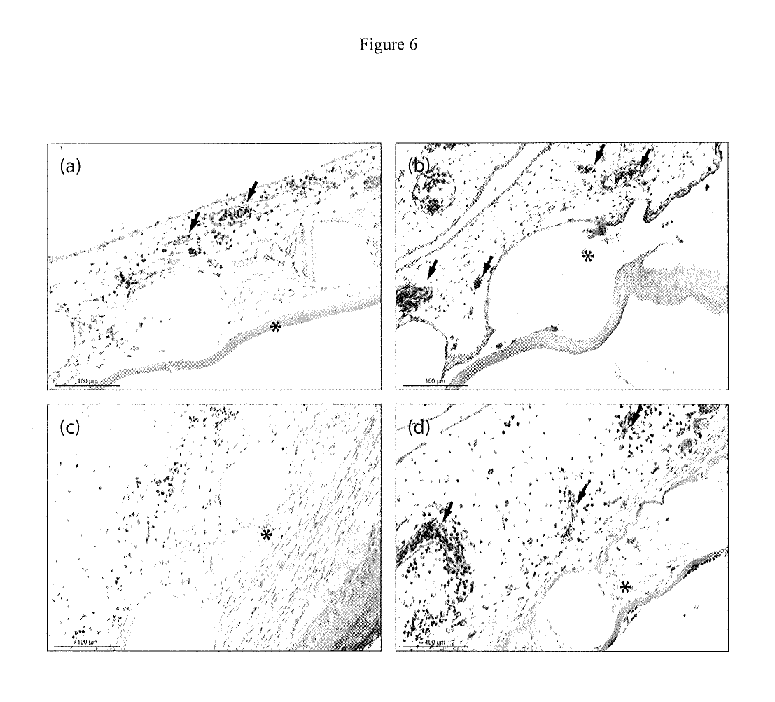

FIG. 7 shows the molecular structures of alginic acid (left) and hyaluronic acid (right).

### DETAILED DESCRIPTION OF THE INVENTION

Conjugation of VEGF inhibitors, including antibodies, to polymers, polysaccharides or other biopolymers is used to extensively reduce clearance of VEGF inhibitors from tissues. In some cases, improvements in binding may also be observed. Conjugates are applied directly to sites for which reductions in VEGF activity provides therapeutic benefit. For example, VEGF inhibitor/biopolymer conjugates may be applied as part of a topical formulation for treatment of neovascular macular degeneration.

Humans produce numerous isoforms of VEGF, including VEGF₁₂₁, VEGF₁₂₁b, VEGF₁₄₅, VEGF₁₆₅, VEGF₁₆₅b, VEGF₁₈₉, and VEGF₂₀₆. To be most effective, a VEGF inhibitor should act on all isoforms, though that level of activity is not required for an inhibitor to be used within the scope of the embodiments of the invention. Conjugation of a VEGF inhibitor to a polymer or polymer construct does not necessarily change the composition or sequence of the inhibitor, unless this was necessitated by the coupling strategy used. However, conjugation of an inhibitor to a polymer or polymer construct could reduce or abolish the affinity of the therapeutic agent for some or all of the VEGF isoforms. For example, higher VEGF isoforms (e.g. VEGF₁₆₅) contain a heparin binding domain, and conjugation of a charged polymer, such as hyaluronic acid, to anti-VEGF could result in loss of antibody affinity for VEGF₁₆₅ due to weaker competing interactions with the pendant hyaluronic acid chain. Those skilled in the art will recognize with the benefit of this disclosure that in some cases retention of binding affinity and biological activities of the therapeutic agent in the construct should often be carefully measured.

VEGF inhibitors can be conjugated to a diversity of macromolecular species. These include, for example, but are not limited to, synthetic polymers, native and chemically modified biopolymers, including those with alkyl or aryl substituents via chemical linkages such as esters or amides, and propylene glycol-functionalized alginates. Cross-linked polymer constructs, either through native binding of divalent ions (e.g. calcium) or through polymerizable groups, such as vinyl or allyl functionality, may also be used.

A number of VEGF-binding moieties could be incorporated with the conjugates described herein. These include but are not limited to monoclonal antibodies (e.g. bevacizumab) (reported, for example, in Ferrara, et al., “Bevacizumab (Avastin®), a humanized anti-VEGF monoclonal antibody for cancer therapy” Biochem Biophys Res Commun. 2005; 333(2):328-35), antibody fragments (e.g. ranibizumab) (reported, for example, in Folk & Stone “Ranibizumab therapy for neovascular age-related macular degeneration” N Engl J Med. 2010; 363(17):1648-55), aptamers (e.g. pegaptanib) (reported, for example, in Ng E. W., et al., “Pegaptanib, a targeted anti-VEGF aptamer for ocular vascular disease” Nat Rev Drug Discov. 2006; 5(2):123-32)), and peptides (reported, for example, in Binetruy-Tournaire, et al., “Identification of a peptide blocking vascular endothelial growth factor (VEGF)-mediated angiogenesis” EMBO J. 2000; 19(7):1525-33. PMCID: 310222).

Molecules that bind or regulate other signaling factors involved in regulating blood vessel formation and maintenance may be incorporated in embodiments of the invention. Constructs composed of native or chemically modified alginates as well as other native or chemically modified polysaccharides besides alginates, such as esterification of a fraction of the carboxylic acid groups on the monomers, may also be used in preparing these constructs. These polysaccharides may include, for example, but are not limited to hyaluronic acid, carboxymethylcellulose, chitosan, fucoidan, dextran and derivatives such as dextran sulfate, pentosan polysulfate, carrageenans, pectins and pectin derivatives, and cellulose derivatives. Other suitable polymers include, but are not limited to, glucosaminoglycans (GAGs) such as dermatan sulfate, chondroitin sulfate, keratan sulfate, heparin, heparan sulfate, and hyaluronan (i.e., hyaluronic acid/hyaluronate). Additional useful hydrophilic polymers include, for example, agarose, dextran, starch, methyl cellulose, poly(ethylene glycol) (“PEG”) (though in some embodiments of the invention PEG may not be used), collagen, gelatin, fibrin, fibrinogen, fibronectin, or vitronectin. Synthetic water-soluble polymers and other related macromolecules may also be used in these conjugates. These include, for example, but are not limited to poly(ethylene oxide), poly(acrylic acid), poly(methacrylic acid), poly(acrylamide), charged polystyrene derivatives, polyvinylpyrrolidone, poly(amino acids), poly(amines), and other polyelectrolytes.

Those skilled in the art will, with the benefit of this disclosure, recognize reactions that could be adapted for performing the conjugation between antibodies and polymers or other macromolecules. These include, for example, Michael-type additions (Oh, et al. “Signal transduction of hyaluronic acid-peptide conjugate for formyl peptide receptor like 1 receptor” Bioconjug Chem. 2008; 19(12):2401-8), disulfide bond formation (Liu, et al. “Disulfide-crosslinked hyaluronan-gelatin sponge: growth of fibrous tissue in vivo” J Biomed Mater Res A. 2004; 68(1):142-9), click reactions (Malkoch, et al. “Synthesis of well-defined hydrogel networks using click chemistry” Chem Commun (Camb). 2006(26):2774-6), formation of a Schiff base (Bhargava, et al. “Synthesis of aminobenzyltriethylenetetraminohexaacetic acid: conjugation of the chelator to protein by an alkylamine linkage” J Protein Chem. 1999; 18(7):761-70, transamination (Scheck, et al. “Optimization of a biomimetic transamination reaction” J Am Chem Soc. 2008; 130(35):11762-70), and amide-bond formation described in this application. Some of these may require prior chemical functionalization of the antibody, polysaccharide, or both.

Embodiments of the invention differ from other reported conjugates in a number of ways. These include the strong chemical dissimilarity of mediators of angiogenesis, e.g. through the presence of heparin-binding domains on certain VEGF isoforms, as compared to mediators of inflammation. Furthermore, while there are fundamental connections between inflammation and angiogenesis, the two processes present distinctly different mechanisms for treatment. In the case of inflammation, if the underlying inflammatory processes can be resolved, the tissue will in most cases move onto phases of healing and repair. However, for conditions characterized by mysregulation of blood vessel formation and maintenance, inhibiting the mediators of angiogenesis only masks an underlying disease state with a propensity for formation of new blood vessels, as in the case of cancerous tumors. This changes the strategy and requirements for localized neutralization of angiogenesis as compared to treating inflammation.

### EXAMPLES

This example provides preparation and use of a VEGF monoclonal antibody conjugated to hyaluronic acid having molecular weight 1.6 MDa. Composition was measured using polyacrylamide gel electrophoresis (PAGE) analysis and fluorescence immunosorbent assay. Binding affinity of the construct was measured using an optical biosensor and compared to that of the unconjugated monoclonal antibody and a conjugate to sodium alginate having molecular weight 100 kDa. Biological activities of the conjugate were assessed using an accepted ex vivo assay.

Materials

HA (˜1.6×10⁶ g/mol), sodium alginate (˜1×10⁵ g/mol), N-hydroxysulfosuccinimide sodium salt (sulfo-NHS), N-(3-dimethyl-aminopropyl)-N′-ethylcarbodiimide hydrochloride (EDC), and 4-(dimethylamino)pyridine (4-DMAP) were purchased from Sigma-Aldrich (St. Louis, Mo.) and used as received. Monoclonal anti-human VEGF antibody and rhVEGF₁₆₅ were purchased from R&D Systems Inc (Minneapolis, Minn.).

### HA-mAb or Alginate-mAb Preparation

The first step reaction was activation of the carboxylic acid groups on the monomers. The active ester intermediate was subsequently used as a precursor for the coupling reaction with anti-hVEGF monoclonal antibody for in vitro and in vivo studies. HA or alginate (10 mg, 6.25 nmol) was dissolved in 1 mL PBS (pH˜7.4). EDC (120 mg, 625 nmol), sulfo-NHS (217 mg, 1 mmole), and 4-DMAP (10 mg) were added as solids to the HA solution and allowed to dissolve and react overnight before adding mAb. Antibodies (0.5 mg) were added to the activated polysaccharide solution. The reaction proceeded at 4° C. overnight. The solution was dialyzed (MW cut-off 300 kDa) using a spin dialysis apparatus purchased from Next Group (Southborough, Mass.) against PBS for 16 hrs with 4 changes of PBS solutions.

### Polyacrylamide Gel Electrophoresis

10 mL of 4% acrylamide/bis-acrylamide solution in 1% TBE buffer was prepared from 40% acrylamide/bis-acrylamide solution (Sigma, Mo.) and 10×TBE buffer (Promega, Wis.). The solution was mix on a stir plate for 10 minutes, and followed by adding 50 μl of 10% (w/v) ammonium persulfate and 4 μl of NNN′N′-tetramethylethylenediamine (Sigma, Mo.). The solution was mixed well and injected into the glass plates (Bio-rad, CA). After loading, 30 minutes to an hour passed until the gel hardened then 5 μl of each of the standards was loaded, which consisted of 0.1% HA, 0.05% HA, 0.025% HA, etc. Samples were loaded at two different concentrations 1× and 0.1× stock solution and 125 V was applied across the gel for 5 hrs.

### Hyaluronan or Alginate Quantification

The gel was stained in 0.5% Alcian Blue (Sigma, Mo.) in 3% Acetic Acid for 45 min followed by destaining with 3% Acetic Acid overnight. The gel image was taken and quantitatively analyzed using Fujifilm LAS-3000 and MultiGauge image analysis software.

### Fluorescence ImmunoSorbent Assay

Immuno 96 MicroWell Plate (NUNC, NY) was first incubated with 50 ml of 2 mg/ml of Rabbit Anti-Mouse IgG (Jackson, Pa.) in PBS each well at 4° C. overnight. The solution was discarded and the plate was washed with detergent for three times followed by incubation of 200 ml of the blocking buffer, which contained 0.25% BSA, 0.05% Tween, and 1 mM EDTA in 1×PBS, at 37° C. for 1 hr. Discard the blocking buffer. The antibody of interest was prepared in carbonate buffer, and the standards were prepared using mouse whole IgG (Jackson, Pa.) in triplicates. 50 ml of each solution was loaded into designated wells followed by 1 hr incubation with shaking at room temperature.

The solutions were discarded and each well was washed by detergent for three times with ten minutes of incubation in between. 2 mg/ml of Goat anti-mouse IgG conjugated with Alexa 488 (Invitrogen, CA) was prepared in carbonate buffer and 50 ml of this solution was loaded into each well followed by one hour incubation with shaking in the dark at room temperature. Wash the wells three times with detergent for 10 min in between and preserve the plate with PBS. The plate was read and analyzed by SAFIRE Microplate Reader with excitation at 488 nm and emission at 520 nm.

### Binding Interaction

Octet system (ForteBio Corp.) was utilized to measure HA-mAb binding interaction. Streptavidin modified sensor tips were hydrated in PBS. All the samples were diluted in PBS. Mouse anti-human VEGF monoclonal antibody and its polysaccharide conjugates were biotinylated with EZ-link Sulfo-NHS-LC-LC-Biotin purchased from Pierce (Rockford, Ill.). The reaction was carried out at 1:1 molar ratio of the biotin linker and antibody for 1 hr in 4° C., followed by 12 hrs of dialysis in 4° C. The biotinylated antibody and polysaccharide-mAb conjugates were diluted to 10 mg/ml in PBS. Recombinant human VEGF₁₆₅ was diluted to desired concentration. The experimental setup is as followed in the following specific sequence: PBS 5 min (baseline), Antibody or polysaccharide-mAb solution 15 min (loading), PBS 5 min (wash), PBS 5 min (baseline), rhVEGF₁₆₅ solution 30 min (association), and PBS 60 min (dissociation). The results were analyzed by the ForteBio analysis program that generated the best fit binding isotherm, and kₒₙ and k_{off} are calculated from the isotherm.

### Ex Vivo Chick CAM Assay and Histology Analysis

To prepare samples for the CAM assay, 2 mg/ml collagen gels were cast on nylon mesh (Sefar Filtration Inc., Depew, N.Y.) for increased maneuverability. Final concentrations of additions to the collagen gel were added as follows: antiVEGF (1:100), HA-antiVEGF (10 mg/ml), HA (10 mg/ml) and VEGF (1 mg/ml). Gels were allowed to polymerize at 37° C. for 40 minutes. White Leghorn eggs were purchased locally from a farm and incubated at 38° C. with 70% humidity in a rotating circulated air incubator (G.Q.F. Manufacturing Co., Savanna, Ga.). Eggs were cracked into Petri dishes on day 3 and placed in a 37° C. incubator (Form a Scientific, Waltham, Mass.). Fibrin gels were placed on the CAM of 10 day old embryos. Gels of each type were placed on the embryo's chorioallantoic membrane. Gels were then harvested 5 days post placement, fixed in paraformaldehyde for parrafin embedding, sectioning and histological manipulations.

Masson trichrome stain was performed using Chromaview staining kit purchased from Richard-Alan Scientific (Kalamazoo, Mich.) and followed the manufacturer's protocol for staining. Basically, the sections were deparaffinized and hydrated, followed by fixation in Bouin's Fluid. The sections were then stained with the following order: Working Weigert's Iron Hematoxylin, Biebrich Scarlet-Acid Fuchsin Solution, Phosphotungstic-Phosphomolybdic Acid, Aniline Blue, and acetic acid. The sections were dehydrated and mounted for imaging analysis. Histological images were taken with Leica DM IL LED microscope system (Germany).

Specific examples tested include monoclonal antibodies against VEGF conjugated to hyaluronic acid or sodium alginate, a naturally occurring polysaccharide derived from seaweed that is used extensively in biomedical applications using the same carbodiimide coupling chemistry described. Alginate is an anionic polysaccharide derived from seaweed. It binds calcium cations avidly, and calcium-crosslinked alginate gels have been showed to be chemically and immunologically inert in vivo. The molecular weight of commercial formulations can be in excess of 600 kDa, and solutions derived from these are highly viscous.

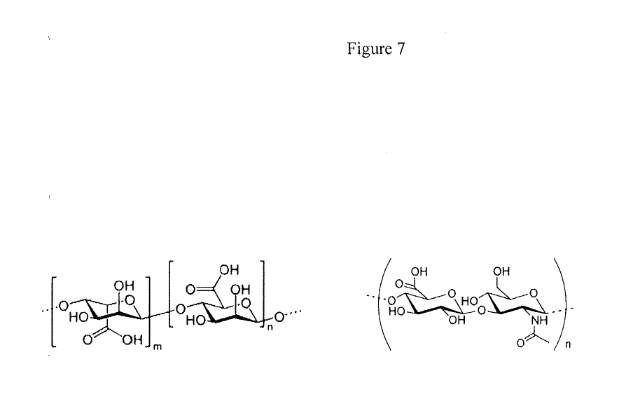

The monomer structures of HA alginic acid (referred to here as alginate) are shown in FIG. 7. For the given structures, n may be in the range of 1-100,000 for all these polysaccharides. In HA, every other cyclic sugar has a carboxylic acid group that is potentially negatively charged at neutral pH, making the effective degree of anionic functionalization 0.5. A diversity of chemical strategies may be used to modify the material or biochemical properties of the final products. For alginate, both the β-D-mannuronate and the α-L-guluronate monomers have a carboxylic acid group, making the degree of anionic functionalization equal to 1.0. The charge density of alginate may play a role in its contribution to binding interactions with VEGF isoforms.

The binding affinities of anti-VEGF, (anti-VEGF)-HA, and (anti-VEGF)-alginate were measured against VEGF 165 using the ForteBio Octet. The results are as follows:

| | K_{D} (M) | kₒₙ (1/MS) | k_{off} (1/s) |
|---|---|---|---|
| anti-VEGF | 4.08E−10 ± 1.41E−10 | 8.60E+04 ± 3.05E+04 | 3.25E−05 ± 3.37E−06 |
| HA-anti-VEGF | 1.10E−10 ± 4.41E−11 | 8.91E+05 ± 1.21E+05 | 9.42E−05 ± 2.30E−05 |
| Alginate-anti- | 1.06E−10 ± 1.41E−11 | 2.06E+06 ± 5.05E+05 | 2.14E−04 ± 3.39E−05 |
| VEGF | | | |

For both polysaccharide conjugates of anti-VEGF, K_{D} demonstrated statistically significant enhancements over the unconjugated anti-VEGF. This suggests that these polysaccharides were able to increase the association rate (kₒₙ) or decrease the dissociation rate (k_{off}), leading to improved binding of VEGF. Such synergistic enhancements are not commonly associated with polymer conjugation. Veronese & Mero, “The impact of PEGylation on biological therapies” BioDrugs. 2008; 22(5):315-29. Despite the affinity of the heparin-binding domain of VEGF₁₆₅, conjugation to charged polysaccharides does not seem to affect the affinity of anti-VEGF for this particular isoform.

Patents, patent applications, publications, scientific articles, books, web sites, and other documents and materials referenced or mentioned herein are indicative of the levels of skill of those skilled in the art to which the inventions pertain, as of the date each publication was written, and all are incorporated by reference as if fully rewritten herein. Inclusion of a document in this specification is not an admission that the document represents prior invention or is prior art for any purpose.

1. A composition comprising:
a hydrophilic polymer; and
a neovascularization-inhibiting ligand-binding moiety covalently attached to the polymer;
wherein the hydrophilic polymer increases residence time of the binding moiety at a site where inhibition of neovascularization is desired relative to residence time of binding moiety without the hydrophilic polymer.

2. The composition of claim 1, wherein the hydrophilic polymer is selected from the group consisting of alginate, hyaluronic acid, carboxymethylcellulose, chitosan, fucoidan, dextran, dextran sulfate, pentosan polysulfate, carrageenans, pectins, pectin derivatives, cellulose derivatives, glucosaminoglycans (GAGs), dermatan sulfate, chondroitin sulfate, keratan sulfate, heparin, heparan sulfate, hyaluronan, agarose, starch, methyl cellulose, poly(ethylene oxide) (“PEO”) or poly(ethylene glycol) (“PEG”), collagen, gelatin, fibrin, fibrinogen, fibronectin, vitronectin, poly(ethylene oxide), poly(acrylic acid), poly(methacrylic acid), poly(acrylamide), charged polystyrene derivatives, polyvinylpyrrolidone, poly(amino acids); poly(amines), poly(acrylic acid), polyelectrolytes; polymer constructs of the foregoing, and micelles of the foregoing, formed of any of these.

3. The composition of claim 2, wherein the hydrophilic polymer is hyaluronic acid.

4. The composition of claim 2, wherein the hydrophilic polymer is poly(ethylene oxide) or poly(ethylene glycol).

5. The composition of claim 1, wherein the binding moiety is selected from the group consisting of a humanized monoclonal antibody, an antibody fragment, a soluble receptor, an aptamer, and a peptide.

6. The composition of claim 1, wherein the binding moiety is selected from the group consisting of a humanized anti-VEGF monoclonal antibody, an anti-VEGF antibody fragment, an anti-VEGF aptamer, and an anti-VEGF peptide.

7. The composition of claim 6, wherein the binding moiety is selected from the group consisting of bevacizumab, ranibizumab, aflibercept, pegaptanib, and biosimilar versions of them.

8. A composition comprising:
a hydrophilic polymer; and
a VEGF-inhibiting moiety covalently attached to said hydrophilic polymer;
wherein the hydrophilic polymer retains the binding moiety at a site where inhibition of neovascularization is desired.

9. The composition of claim 8, wherein the VEGF-inhibiting binding moiety is selected from the group consisting of bevacizumab, ranibizumab, aflibercept, and pegaptanib.

10. A composition comprising:
a hydrophilic polymer, wherein said hydrophilic polymer is hyaluronic acid; and bevacizumab covalently attached to said hydrophilic polymer.

11. The composition of claim 1, wherein the polymer is uncrosslinked.

12. The composition of claim 1 wherein the composition further comprises a substance further enhancing retention at a site of application.

13.-14. (canceled)

15. A method of treatment comprising;
locally administering to a disorder site of a patient in need of treatment a composition of claim 1.

16. The method of claim 12, wherein the disorder site is an eye exhibiting wet macular degeneration.

17. A method for increasing binding affinity of an anti-VEGF antibody, comprising covalently bonding an anti-VEGF antibody with a polysaccharide.

18. A method for increasing binding affinity of a ligand-binding moiety that inhibits neovascularization, comprising covalently bonding a said ligand-binding moiety with a hydrophilic polymer.

19. The method of claim 18, wherein said hydrophilic polymer is selected from the group consisting of alginate, hyaluronic acid, carboxymethylcellulose, chitosan, fucoidan, dextran, dextran sulfate, pentosan polysulfate, carrageenans, pectins, pectin derivatives, cellulose derivatives, glucosaminoglycans (GAGs), dermatan sulfate, chondroitin sulfate, keratan sulfate, heparin, heparan sulfate, hyaluronan, agarose, starch, methyl cellulose, poly(ethylene oxide) (“PEO”) or poly(ethylene glycol) (“PEG”), collagen, gelatin, fibrin, fibrinogen, fibronectin, vitronectin, poly(ethylene oxide), poly(acrylic acid), poly(methacrylic acid), poly(acrylamide), charged polystyrene derivatives, polyvinylpyrrolidone, poly(amino acids); poly(amines), poly(acrylic acid), polyelectrolytes; polymer constructs of the foregoing, and micelles of the foregoing, formed of any of these.
